(19) United States
(12) Patent Application Publication
(10) Pub. No.: US 2008/0294039 A1
(43) Pub. Date: Nov. 27, 2008

(54) Assembly with hemostatic and radiographically detectable pellets

(75) Inventors: Michael L. Jones, San Clemente, CA (US); Paul Lubock, Laguna Niguel, CA (US)

Correspondence Address: EDWARD J. LYNCH;Patent Attorney, Suite 562, One Embarcadero Center, San Francisco, CA, 94111, US

(73) Assignee: SenoRx, Inc.

(21) Appl. No.: 12/150,649

(22) Filed: Apr. 29, 2008

Related U.S. Application Data
(63) Continuation-in-part of application No. 11/881,264, filed on Jul. 26, 2007.
(60) Provisional application No. 60/835,740, filed on Aug. 4, 2006.

(51) Int. Cl. A61B 6/00

(52) U.S. Cl. 600/431

(57) ABSTRACT

The remotely imagable pellet system described has a plurality of pellets disposed within a delivery tube or cannula that are formed at least in part of a polysaccharide such as starch and at least one radiographically detectable bioabsorbable pellet which preferably has a radiopaque element. The radiographically detectable bioabsorbable pellet has at least one and preferably two pellets proximal and distal thereto which are formed of polysaccharide.

### RELATED APPLICATIONS

This application is a continuation-in-part of application Ser. No. 11/881,264, filed on Jul. 26, 2007, which claims priority from provisional application Ser. No. 60/835,740, filed on Aug. 4, 2006, all of which are incorporated herein by reference in their entirety and from which priority is claimed.

### FIELD OF THE INVENTION

The invention is generally directed to remotely detectable, intracorporeal marker pellets and devices and methods for the delivery of such marker pellets to a desired location within a patient's body such as a biopsy or lumpectomy site.

### BACKGROUND OF THE INVENTION

In diagnosing and treating certain medical conditions, it is often desirable to mark a suspicious body site for the subsequent taking of a biopsy specimen, for delivery of medicine, radiation, or other treatment, for the relocation of a site from which a biopsy specimen was taken, or at which some other procedure was performed. As is known, obtaining a tissue sample by biopsy and the subsequent examination are typically employed in the diagnosis of cancers and other malignant tumors, or to confirm that a suspected lesion or tumor is not malignant. The information obtained from these diagnostic tests and/or examinations is frequently used to devise a therapeutic plan for the appropriate surgical procedure or other course of treatment.

In many instances, the suspicious tissue to be sampled is located in a subcutaneous site, such as inside a human breast. To minimize surgical intrusion into a patient's body, it is often desirable to insert a small instrument, such as a biopsy needle, into the body for extracting the biopsy specimen while imaging the procedure using fluoroscopy, ultrasonic imaging, x-rays, magnetic resonance imaging (MRI) or any other suitable form of remote imaging technique or palpation. Examination of tissue samples taken by biopsy is of particular significance in the diagnosis and treatment of breast cancer. In the ensuing discussion, the biopsy and treatment site described will generally be the human breast, although the invention is suitable for marking biopsy sites in other parts of the human and other mammalian body as well.

Periodic physical examination of the breasts and mammography are important for early detection of potentially cancerous lesions. In mammography, the breast is compressed between two plates while specialized x-ray images are taken. If an abnormal mass in the breast is found by physical examination or mammography, ultrasound may be used to determine whether the mass is a solid tumor or a fluid-filled cyst. Solid masses are usually subjected to some type of tissue biopsy to determine if the mass is cancerous.

If a solid mass or lesion is large enough to be palpable, a tissue specimen can be removed from the mass by a variety of techniques, including but not limited to open surgical biopsy, a technique known as Fine Needle Aspiration Biopsy (FNAB) and instruments characterized as “vacuum assisted large core biopsy devices”.

If a solid mass of the breast is small and non-palpable (e.g., the type typically discovered through mammography), a vacuum assisted large core biopsy procedure is usually used. In performing a stereotactic biopsy of a breast, the patient lies on a special biopsy table with her breast compressed between the plates of a mammography apparatus and two separate x-rays or digital video views are taken from two different points of view. A computer calculates the exact position of the lesion as well as depth of the lesion within the breast. Thereafter, a mechanical stereotactic apparatus is programmed with the coordinates and depth information calculated by the computer and such apparatus is used to precisely advance the biopsy needle into the small lesion. The stereotactic technique may be used to obtain histologic specimens. Usually at least five separate biopsy specimens are obtained from locations around the small lesion as well as one from the center of the lesion.

The available treatment options for cancerous lesions of the breast include various degrees of mastectomy or lumpectomy, radiation therapy, chemotherapy and combinations of these treatments. However, radiographically visible tissue features, originally observed in a mammogram, may be removed, altered or obscured by the biopsy procedure, and may heal or otherwise become altered following the biopsy. In order for the surgeon or radiation oncologist to direct surgical or radiation treatment to the precise location of the breast lesion several days or weeks after the biopsy procedure was performed, it is desirable that a biopsy site marker be placed in the patient's body to serve as a landmark for subsequent location of the lesion site. A biopsy site marker may be a permanent marker (e.g., a metal marker visible under x-ray examination), or a temporary marker (e.g., a bioresorbable marker detectable with ultrasound). While current radiographic type markers may persist at the biopsy site, an additional mammography generally must be performed at the time of follow up treatment or surgery in order to locate the site of the previous surgery or biopsy. In addition, once the site of the previous procedure is located using mammography, the site must usually be marked with a location wire which has a hook on the end which is advanced into site of the previous procedure. The hook is meant to fix the tip of the location wire with respect to the site of the previous procedure so that the patient can then be removed from the confinement of the mammography apparatus and the follow-up procedure performed. However, as the patient is removed from the mammography apparatus, or otherwise transported the position of the location wire can change or shift in relation to the site of the previous procedure. This, in turn, can result in follow-up treatments being misdirected to an undesired portion of the patient's tissue.

As an alternative or adjunct to radiographic imaging, ultrasonic imaging (herein abbreviated as “USI”) or visualization techniques can be used to image the tissue of interest at the site of interest during a surgical or biopsy procedure or follow-up procedure. USI is capable of providing precise location and imaging of suspicious tissue, surrounding tissue and biopsy instruments within the patient's body during a procedure. Such imaging facilitates accurate and controllable removal or sampling of the suspicious tissue so as to minimize trauma to surrounding healthy tissue.

For example, during a breast biopsy procedure, the biopsy device is often imaged with USI while the device is being inserted into the patient's breast and activated to remove a sample of suspicious breast tissue. As USI is often used to image tissue during follow-up treatment, it may be desirable to have a marker, similar to the radiographic markers discussed above, which can be placed in a patient's body at the site of a surgical procedure and which are visible using USI. Such a marker enables a follow-up procedure to be performed without the need for traditional radiographic mammography imaging which, as discussed above, can be subject to inaccuracies as a result of shifting of the location wire as well as being tedious and uncomfortable for the patient.

Placement of a marker or multiple markers at a location within a patient's body requires delivery devices capable of holding markers within the device until the device is properly situated within a breast or other body location. Accordingly, devices and methods for retaining markers within a marker delivery device while allowing their expulsion from the devices at desired intracorporeal locations are desired.

In addition to marking functions, frequently it is desirable to provide treatments with the marker members such as hemostatic treatment and the like.

### SUMMARY OF THE INVENTION

The invention is generally directed to a remotely imagable pellet system suitable for deployment at a site within a patient's body, particularly a biopsy or lumpectomy site such as in a patient's breast from which tissue has been removed. The imagable pellet system has a plurality of hemostatic pellet members formed of material such as a polysaccharide such as starch (e.g. cornstarch) sufficient to accelerate thrombus formation at the site and one or more radiographically detectable pellets. The radiographically detectable pellets preferably have a radiopaque element to make the pellet radiographically detectable. The radiopaque element is preferably non-magnetic to facilitate or to not otherwise interfere with magnetic resonance imaging (MRI). The radiographically detectable pellet(s) are preferably at least in part formed of bioabsorbable polymeric material. The hemostatic pellets reduce bleeding at the site and in turn minimize development of hematoma.

Suitable polysaccharides, such as starch have molecular weights of about 3500 to about 200,000 Daltons and the pellets are preferably formed from a dry powder thereof having a particle size of about 20-100 micrometers. The pellets formed of starch or other polysaccharide may be formed of compressed powder. The polysaccharide pellets rapidly absorb fluid and hydrate and in the process dehydrate blood at the site of deployment to rapidly initiate clotting and ultimately hemostasis. One or more of the hemostatic pellets may be coated with a bioabsorbable material to provide short term control of hemostasis at the site.

At least one of the pellets is a remotely detectable bioabsorbable marker body which preferably is radiographically detectable (e.g. includes a radiopaque element connected thereto or incorporated therein) to provide long term identification of the intracorporeal site in which the pellet is placed. Preferably, the radiopaque element is formed of non-magnetic material such as titanium, platinum, gold, iridium, tantalum, tungsten, silver, rhodium, non-magnetic stainless steel (316) and the like. The radiopaque element should have a shape and a maximum dimension of about 0.5 to about 5 mm, preferably about 1 to about 3 mm to ensure remote identification, particularly with MRI. Typical shapes for the radiopaque elements are Ω (e.g. for stainless steel) or S (e.g. for titanium).

The polysaccharide pellets will generally be about 0.2 to about 3 mm, preferably about 1 to about 2 mm, in diameter and about 3 to about 7 mm, preferably about 4 to about 6 mm in length. Typically, the length is 6.1 mm and the diameter is 1.4 mm. Preferably, the starch pellet is formed of a mixture of about 50-85% (wt %) corn starch (USP) and about 15-50% (wt. %) methylcellulose and typically is a mixture of about 65% starch and 35% methylcellulose.

The bioabsorbable radiographically detectable pellet will generally be about 0.5 to about 4 mm, preferably about 1 to about 3 mm, in diameter and about 1.5 to about 6 mm, preferably about 2 to about 5 mm in length. The bioabsorbable pellet is formed of synthetic polymeric materials such as polyglycolic acid (PGA), polylactic acid polycaprolactone and copolymers thereof. Polymers may also be formed of dehydrated dimers, e.g. glycolide and lactide. The ratio of polymers can be varied to adjust the properties of the final polymers in a conventional manner. Preferably, the polymer is a copolymer of polylactic acid and polyglycolic acid in a weight ratio of about 65% to 35%.

The system preferably has a pusher pellet at the proximal most position within the delivery tube which is formed of bioabsorbable polymeric material such as polyethylene glycol and which is slightly larger than the more distal starch or polysaccharide pellets. The pusher pellet is the about the same size as the radiographically detectable bioabsorbable pellet and should have sufficient strength properties to avoid deformation when pushing the other pellets out of the delivery tube into the intracorporeal site.

The plurality of pellets embodying features of the invention can be readily delivered to the desired location by suitable delivery systems such as disclosed in co-pending application Ser. No. 10/444,770, filed on May 23, 2003 and Ser. No. 10/753,277, filed on Dec. 23, 2003. The marker delivery system generally has an elongated cannula or syringe-like body with proximal and distal ports and an inner lumen extending between the ports. The hemostatic and radiographically detectable pellets are slidably disposed within the inner lumen of the delivery cannula and a plunger for moving the pellets is slidably disposed within the inner lumen of the delivery cannula proximal to the pellets. The plunger is movable from an initial position proximal to the markers within the tube, to a delivery position close to the discharge opening in the distal end of the cannula to push the marker members out of the discharge opening into the target tissue site. Preferably, the radiographically detectable pellet has one or more hemostatic pellets proximal and one or more hemostatic pellets distal thereto within the inner lumen of the delivery cannula.

Upon being discharged into the intracorporeal target site, the hemostatic pellets quickly take up water from body fluid at the site and initiate the clotting process. The at least one radiographically detectable pellet at the site enables short term detection (at least three weeks, preferably at least four weeks but less than a year) by remote USI and preferably long term detection by remote mammographic imaging or MRI identification.

The cannula of the marker delivery device may be configured to fit within the guide cannula of a biopsy device, such as the SenoCor 360™ biopsy device sold by SenoRx (the present assignee), the EnCor,™ biopsy device sold by SenoRx, the Mammotome® (sold by Johnson & Johnson) or a coaxial needle guide. The delivery cannula can also be configured to fit into the proximal end of a tubular cutting element such as found in the EnCor™ biopsy system sold by SenoRx which is the subject of co-pending application Ser. No. 10/911,106, filed on Aug. 3, 2004.

A variety of therapeutic or diagnostic agents may be incorporated into the hemostatic and radiographically detectable pellets. Incorporated agents can include for example, additional hemostatic agents to form thrombus at the intracorporeal site, anesthetic agents to control pain, chemotherapeutic agents for treating residual neoplastic tissue or coloring agents to facilitate subsequent visual location of the site. Antibiotics, antifungal agents and antiviral agents may also be incorporated into the fibrous marker. The radiographically detectable pellet(s) may be radioactive seeds to provide irradiation at the site, e.g. from which tissue has been removed.

These and other advantages of the invention will become more apparent from the following detailed description of embodiments when taken in conjunction with the accompanying exemplary drawings.

### BRIEF DESCRIPTION OF THE DRAWINGS

FIG. 1 is a partly cut-away perspective view of a marker delivery assembly embodying features of the invention.

FIG. 2 is a transverse cross-sectional view of the marker delivery assembly of FIG. 1 taken at line 2-2.

FIG. 3 is a transverse cross-sectional view of the marker delivery assembly of FIG. 1 taken at line 3-3.

FIG. 4 is a perspective view, partially in section, of a human breast from which a biopsy specimen has been removed, showing pellets of the assembly shown in FIG. 1 delivered to the biopsy site.

FIG. 5 is a transverse cross-sectional view of a marker member having an outer surface or capsule to retard hemostasis.

### DETAILED DESCRIPTION OF EMBODIMENTS OF THE INVENTION

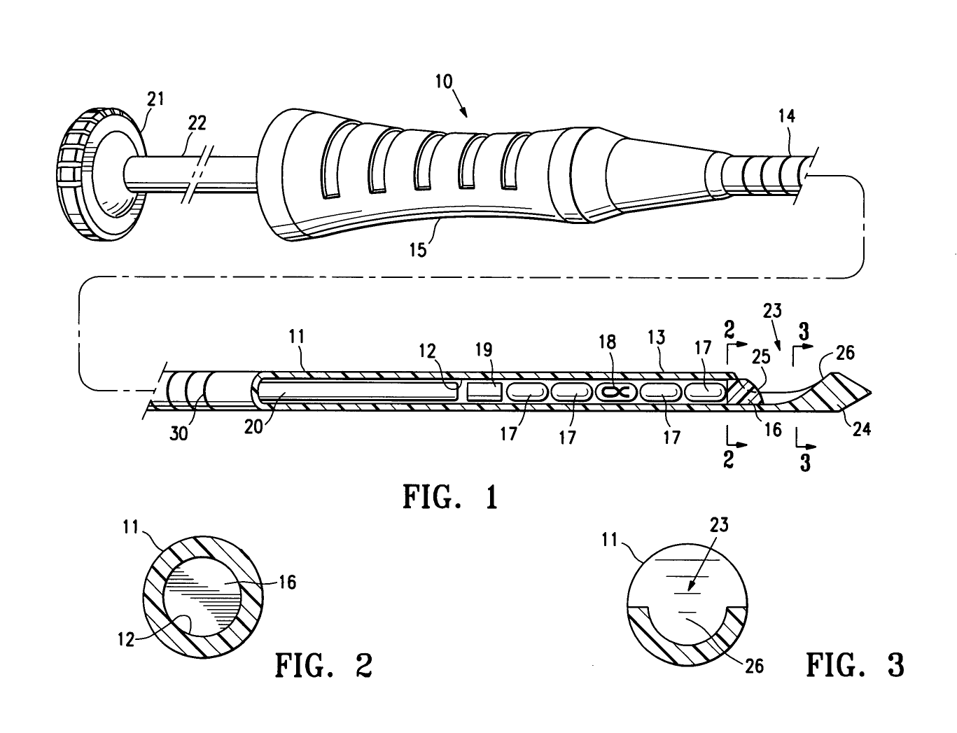

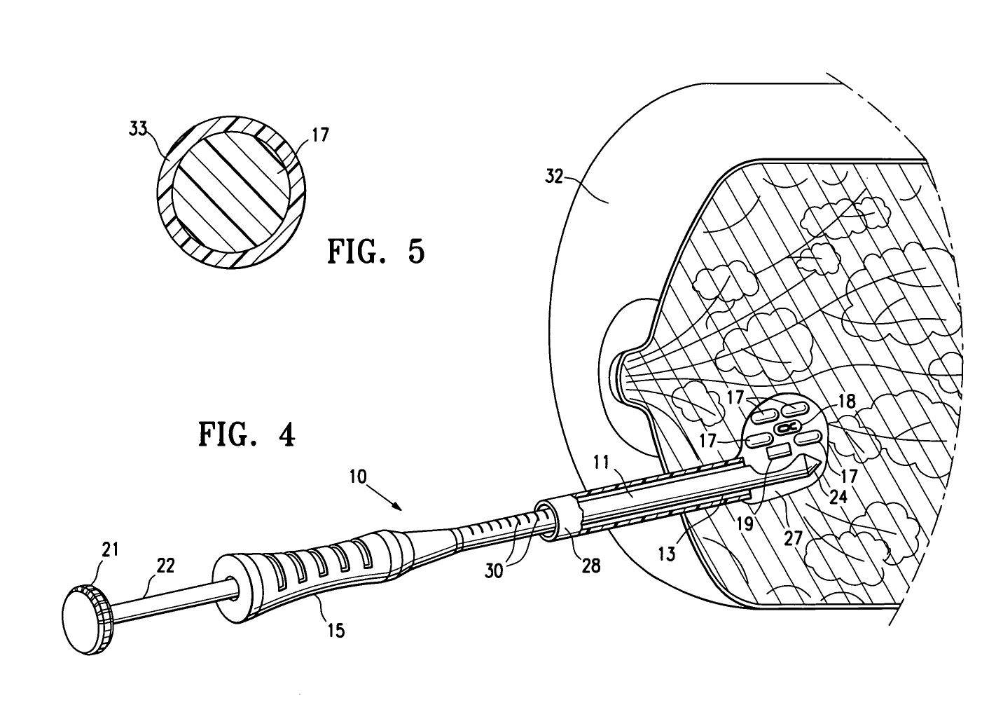

FIG. 1 illustrates marker delivery system 10 embodying features of the invention which include a delivery tube or cannula 11 with an inner lumen 12, a distal portion 13, and a proximal portion 14 with a handle 15. A releasable distal plug 16, two pairs of hemostatic pellets 17 formed of polysaccharide (e.g. starch) and a radiographically detectable marker pellet 18 between the two pairs of pellets 17 are shown disposed within the inner lumen 12. A pusher pellet 19 is located proximal to the pellets 17 and 18. A plunger 20 is slidably disposed within the inner lumen 12 and is provided with a head 21 on the proximal end 22 to allow an operator to press the plunger further into the inner lumen 12 and push the releasable plug 16, the pellets 17 and 18 and the pusher pellet 19 out of the discharge port or opening 23 in the distal end 24 of delivery cannula 11. Cannula handle 15 allows an operator to hold the cannula steady while pressing plunger 20 to discharge the pellets, e.g. into a patient's breast such as shown in FIG. 4.

Releasable plug 16 preferably blocks only a portion of the discharge opening 23 but may substantially fill or occlude the discharge opening. The exposed face 25 of plug 16 is preferably provided with an inclined configuration and is configured to be tight enough, e.g. press fit, in the inner lumen 12 to prevent its inadvertent release which might allow premature discharge of one or more pellets 17 and 18 from delivery cannula 10, but the plug 16 must be easily released when the plunger 20 is pressed deeper into the inner lumen 12 for pellet discharge. An adhesive or mechanical element(s) may be used to hold the releasable plug 16 in a position within the inner lumen 12. Suitable adhesives include polyurethane or polyacrylic based adhesives, polyhydroxymethacrylate base adhesives, fibrin glue (e.g., Tisseal™), collagen adhesive, or mixtures thereof. Suitable mechanical means for securing the releasable plug 16 are described in co-pending application Ser. No. 10/174,401, Jun. 17, 2002. The distal portion 13 of the delivery cannula 11 is provided with a ramp 26 which guides the discharged plug 16 and pellet members 17 and 18 out of the side port 23 into the target site 27 as shown in FIG. 4. The distal tip 24 may be tapered for delivery through a guide tube 28.

The delivery cannula 11 may be provided with markings 30 which serve as visual landmarks to aid an operator in accurately placing the distal portion 13 of the cannula in a desired location 28 within a patient's body for discharging the pellets 16-19.

The exterior of the delivery cannula 11 is preferably configured to fit within a guide cannula sized to accept a SenoCor®, or EnCor™, Mammotome® or Tru-Cut®, biopsy device. Typically, plug 16 and pellets 17, 18 and 19 will have diameters determined by the size of the inner lumen 12 and typically will be about 0.02 inch (0.5 mm) to about 0.5 inch (12 mm), preferably about 0.04 inch (1 mm) to about 0.3 inch (8 mm). Plug 16 may have slightly larger transverse dimensions to provide a tight fit.

FIG. 4 schematically illustrates the delivery of pellets 16, 17 and 18 into a cavity 27 such as a biopsy site in a patient's body. The distal portion 13 of the cannula 11 is shown inserted into a breast 32 through a guide cannula 28 until the distal end is disposed in the cavity 27 where a tissue specimen has been removed. While an operator holds the system 10 by the handle 15 of the delivery tube 11, the plunger 20 is pressed further into the inner lumen 12 of delivery tube to discharge the releasable plug 16 and pellets 17, 18 and 19 into the cavity 27.

The amount of suitable polysaccharide incorporated into the hemostatic pellets 17 is sufficient to cause rapid clotting upon delivery to an intracorporeal site. Typically, the minimum amount of starch to water to form a suitable gel is at least 5%, preferably at least about 10% (wt %). See the description of marker pellets in co-pending application Ser. No. 10/444,770, filed on May 23, 2003 and Ser. No. 10/753,277, filed on Dec. 23, 2003.

As shown in FIG. 5, the hemostatic pellet 17 formed of starch or other suitable polysaccharide may have a protective coating 33 which retards taking up fluid until deployed well into the intracorporeal site. The coating may be a gelatin or suitable bioabsorbable polymer such as polyethylene glycol, polyvinylpyrrolidone, and polyvinyl alcohol among others which proved short term protection to the pellet but are rapidly absorbed at the site to unduly delay hemostasis.

The biopsy site marker assembly embodying features of the invention is a sterile, preferably single use device, which is intended for marking a biopsy site immediately following removal of tissue during percutaneous breast biopsy procedures. The device is comprised of a disposable applicator typically containing four (4) biodegradable starch pellets, one (1) polylactic/polyglycolic acid-based co-polymer (PLA/PGA) pellet with either a stainless steel or titanium embedded wireform and one (1) polyethylene glycol (PEG) push pellet which urges the other pellets out of the device. The deployment device, or applicator, consists of a syringe-type ABS handle attached to a flexible polymeric tube (e.g. Pebax) housing the pellets.

While one or more particular forms of the invention have been illustrated and described herein in the context of a breast biopsy site, it will be apparent that the device and methods having features of the invention may find use in a variety of locations and in a variety of applications, in addition to the human breast, where tissue has been removed. For example, the pellet assembly can be employed at prostate sites to deliver radioactive seeds for irradiation of surrounding tissue and hemostatic pellets for restricting or stopping blood flow at the site. Moreover, various modifications can be made without departing from the spirit and scope of the invention. Accordingly, it is not intended that the invention be limited to the specific embodiments illustrated. It is therefore intended that this invention to be defined by the scope of the appended claims as broadly as the prior art will permit, and in view of the specification if need be. Moreover, those skilled in the art will recognize that features shown in one embodiment may be utilized in other embodiments. Additional details of pellet members and delivery systems may be found in co-pending application Ser. No. 10/753,694, filed on Jan. 7, 2004, Ser. No. 10/976,138, filed on Oct. 27, 2004, and Ser. No. 11/881,264, filed on Jul. 26, 2007.

Terms such as “element”, “member”, “device”, “section”, “portion”, “step”, “means” and words of similar import when used in the following claims shall not be construed as invoking the provisions of 35 U.S.C. §112(6) unless the following claims expressly use the term “means” followed by a particular function without specific structure or expressly use the term “step” followed by a particular function without specific action. All patents and patent applications referred to above are hereby incorporated by reference in their entirety.

What is claimed is:

1. A remotely imagable pellet system for an intracorporeal site, comprising a plurality of pellets which exhibit hemostatic properties at the intracorporeal site and at least one radiographically detectable pellet.

2. The imagable system of claim 1 wherein the pellets exhibiting hemostatic properties are formed at least in part of a bioabsorbable starch or other suitable polysaccharide.

3. The imagable system of claim 1 wherein the radiographically detectable pellet is formed at least in part of bioabsorbable polymeric material.

4. The imagable system of claim 1 wherein the bioabsorbable polymeric material is selected from the group consisting of polyglycolic acid, polylactic acid, polycaprolactone and copolymers and blends thereof.

5. The imagable system of claim 1 wherein the radiographically detectable pellet has a radiopaque element to facilitate relocation of the intracorporeal site.

6. The imagable system of claim 5 wherein the radiopaque element is disposed within the radiographically detectable pellet.

7. The imagable system of claim 5 wherein the radiopaque element is non-magnetic.

8. The imagable system of claim 5 wherein the radiopaque element is formed of a metal selected from the group consisting of titanium, platinum, gold, iridium, tantalum, tungsten, silver, rhodium and non-magnetic stainless steel.

9. The imagable system of claim 1 wherein the radiographically detectable pellet is at least in part a radioactive seed element for irradiation of tissue at the intracorporeal site.

10. The imagable system of claim 1 wherein the hemostatic pellet is coated at least in part by a bioabsorbable polymer to control hemostasis at the intracorporeal site.

11. The imagable system of claim 10 wherein the coating is selected from the group consisting of a gelatin, polyethylene glycol, polyvinylpyrrolidone and polyvinyl alcohol to provide short term protection to the hemostatic pellet but is rapidly absorbed at the site so as to not unduly delay hemostasis.

12. An intracorporeal pellet delivery system, comprising:
a. an elongated tubular shaft which has a distal end, a proximal end, an inner lumen extending between the proximal and distal ends and a discharge opening in a distal shaft section;
b. a plurality of pellet members slidably disposed in the inner lumen of the shaft, which exhibit hemostatic properties at the intracorporeal site;
c. at least one radiographically detectable pellet member disposed in the inner lumen, facilitating subsequent location of the intracorporeal site; and
d. a plunger element which is slidably disposed in part within the inner lumen of the tubular shaft proximal to the pellets therein and which is configured to urge the pellets out the discharge opening in the distal shaft section of the elongated tubular shaft and into the intracorporeal site.

13. The intracorporeal site marker delivery system of claim 12 wherein the pellets exhibiting hemostatic properties are formed at least in part of suitable polysaccharide.

14. The intracorporeal site marker delivery system of claim 13 wherein the polysaccharide is starch.

15. The intracorporeal site marker delivery system of claim 12 wherein a pusher pellet is provided within the inner lumen of the shaft proximal the polysaccharide pellets and proximal to the at least one radiographically detectable bioabsorbable pellet to facilitate advancement of the pellets therein.

16. The intracorporeal site marker delivery system of claim 12 wherein the at least one radiographically detectable bioabsorbable pellet within the inner lumen of the shaft has at least pellet exhibiting hemostatic properties proximal thereto and at least one pellet exhibiting hemostatic properties distal thereto.

17. The intracorporeal site marker delivery system of claim 12 wherein at least two pellets exhibiting hemostatic properties are provided proximal to the radiographically detectable bioabsorbable pellet.

18. The intracorporeal site marker delivery system of claim 12 wherein at least two pellets exhibiting hemostatic properties are provided distal to the radiographically detectable bioabsorbable pellet.

19. The intracorporeal site marker delivery system of claim 12 wherein a pusher pellet is provided within the inner lumen proximal to the pellets which exhibit hemostatic properties and the radiographically detectable bioabsorbable pellet.

20. The intracorporeal site marker delivery system of claim 12 wherein the distal end of the elongated shaft is partially blocked by a releasable plug.

21. A method for delivering pellets to an intracorporeal site within a patient from which tissue has been removed or separated from surrounding tissue and to restrict or stop blood flow therefrom, comprising:
a. providing a delivery device for delivery of a plurality of pellets which includes;
i. an elongated shaft which has an inner lumen, a discharge opening in a distal portion of the elongated shaft;
ii. a plurality of pellets which exhibit hemostatic properties and at least one radiographically detectable pellet which are slidably disposed within the inner lumen of the elongated shaft; and
iii. a plunger element which is slidably disposed within the inner lumen of the marker delivery device proximal to the pellets disposed therein and which is configured to urge the pellets out the discharge opening in the distal portion of the elongated shaft;
b. advancing the delivery device within the patient until the distal end of the delivery device is disposed at the target tissue site and the discharge opening of the delivery device is aligned for marker member deployment; and
c. pressing the plunger element of the delivery device to eject the pellets through the discharge opening in the delivery device and into the intracorporeal site.

22. The method of claim 21 wherein the pellets exhibiting hemostatic properties are formed at least in part of a polysaccharide.

23. The method of claim 21 wherein the radiographically detectable pellet has a radiopaque element.
